(19) United States
(12) Patent Application Publication
(10) Pub. No.: US 2008/0081052 A1
(43) Pub. Date: Apr. 3, 2008

(54) Topical compositions containing solubilized allantoin and related methods

(76) Inventor: Jerry Zhang, Grayslake, IL (US)

Correspondence Address: JERRY ZHANG, 1061 CHADWICK DR., GRAYSLAKE, IL, 60030, US

(21) Appl. No.: 11/542,326

(22) Filed: Oct. 2, 2006

(51) Int. Cl. A61K 31/4172; A61K 9/00

(52) U.S. Cl. 424/400; 514/389

(57) ABSTRACT

A method for enhancing solubility of allantoin in an aqueous vehicle using a solubilization enhancer is described. Aqueous compositions containing the solubilized allantoin at a level of 0.5% by weight or higher are able to be obtained by using urea or urea derivatives as the solubilization enhancer. The aqueous topical compositions are suitable for cosmetic, dermatological, and pharmaceutical use.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

This invention relates to topical compositions for cosmetic, dermatological and pharmaceutical use. In particular, the invention relates to aqueous compositions containing solubilized allantoin as an active ingredient.

### BACKGROUND OF THE INVENTION

Allantoin, known for its therapeutic action on skin, has been widely used for decades in cosmetic and over-the-counter (OTC) topical formulations. It is also used in the topical pharmaceutical applications in skin ulcer therapy, psoriasis medications and analgesic gels.

For example, U.S. Pat. No. 6,864,274 discloses an allantoin-containing skin cream. The disclosed cream is a heterogeneous emulsion system containing an oil phase dispersed in an aqueous phase. Allantoin is dispersed into the formulation from the aqueous phase. U.S. Pat. No. 6,355,259 discloses a method of using allantoin along with a citrate-phosphate buffer system to suppress odor from urea in skin care compositions.

Allantoin has been classified by the Food and Drug Administration (FDA) OTC Topical Analgesic Review Panel as a Category I (safe and effective) active ingredient skin protectant at a level of 0.5% to 2%.

Although allantoin has been used extensively in cosmetic, dermatological, and pharmaceutical applications, the solubility of allantoin at the FDA-approved levels is an issue. For example, solubility of allantoin in water (expressed in g/100 g water @ 25.degree. C.) is 0.45%, which is lower than the FDA-approved levels. Due to this solubility limitation, allantoin is generally added to a topical formulation in a suspended form. Good agitation is generally required to thoroughly disperse the allantoin to achieve suitable suspension.

For use as a cosmetic skin protectant or treatment of many dermatological and mucosal disorders, it is often preferable to use water-based formulations containing solubilized active ingredients, such as a solution, spray or gel, rather than a cream, lotion or an ointment. Creams, lotions, (typically oil-in-water emulsions) and ointments (typically petroleum jelly based compositions) are often comedogenic, acnegenic, or less cosmetically appealing to patients. Furthermore, active ingredient is generally more bioavailable in solubilized form than in insoluble or suspended form. Solubilized allantoin at the FDA-approved levels may be effectively used as a topical skin protectant and for treatment of dermatological and mucosal disorders. Allantoin-containing compositions may be beneficial in promoting wound cleansing and healing.

Accordingly, there is a need for aqueous allantoin compositions where the active ingredient is solubilized at a level of 0.5% by weight or higher not merely suspended for maximum efficacy.

### SUMMARY OF THE INVENTION

It has been unexpectedly discovered that the presence of urea or a urea derivative or any suitable combination of urea and urea derivatives as a solubilization enhancer increases the aqueous solubility of allantoin when compared to the solubility of allantoin in the absence of such a solubilization enhancer. Physically stable, aqueous compositions containing the solubilized allantoin at a level of 0.5% by weight or higher are able to be obtained by using urea or a urea derivative or any suitable combination of urea and urea derivatives as the solubilization enhancer.

Generally speaking, in accordance with the present invention, a method is provided for enhancing solubility of allantoin in an aqueous vehicle using a solubilization enhancer. The solubilization enhancer is a pharmaceutically acceptable organic compound.

Allantoin topical compositions comprise a mixture of the solubilized allantoin at a level of 0.5% by weight or higher and solubilization enhancer in the aqueous vehicle. The compositions are in the form of a solution, spray or gel.

Accordingly, it is an object of the invention to provide a method for enhancing solubility of allantoin to a level of 0.5% by weight or higher in an aqueous vehicle by using a solubilization enhancer.

Another object of the invention is to provide a method of formulating aqueous topical compositions containing solubilized allantoin at a level of 0.5% by weight or higher by using a solubilization enhancer.

A further object of the invention is to formulate aqueous topical compositions comprising a mixture of solubilized allantoin and solubilization enhancer in an aqueous vehicle, where the presence of the solubilization enhancer increases the solubility of allantoin in the aqueous vehicle when compared to the solubility of allantoin in the absence of the solubilization enhancer, and the compositions are physically stable.

Still other objects and advantages of the invention will, in part, be obvious and will, in part, be apparent from the following detailed description of the preferred embodiments.

### BRIEF DESCRIPTIONS OF THE DRAWINGS

FIG. 1 shows general structure of a urea derivative.

FIG. 2 shows general structure of a mono-substituted alkyl urea.

FIG. 3 shows general structure of a mono-substituted hydroxyalkyl urea.

FIG. 4 shows general structure of a mono-substituted carboxyl urea.

### DETAILED DESCRIPTION OF THE PREFERRED EMBODIMENTS

In the disclosed embodiments, a method for enhancing solubility of allantoin in an aqueous vehicle using a solubilization enhancer is described. Aqueous topical compositions comprise a mixture of the solubilized allantoin and solubilization enhancer in the aqueous vehicles at allantoin level of 0.5% by weight or higher.

The term ‘allantoin’, when used in accordance with the present invention, means allantoin either prepared from synthetic method or isolated from natural source (e.g., from comfrey extract), either in admixture or in pure or substantially pure form. Allantoin occurs as a tautomeric mixture, the keto and enol forms being in equilibrium. All physical forms of allantoin, crystalline, semi-crystalline, and amorphous, are contemplated and within the scope of the present invention, either in admixture or in pure or substantially pure form. Furthermore, the stereoisomers of allantoin are also contemplated and within the scope of the present invention. The definition of allantoin in accordance with the present invention embraces all possible stereoisomers and their mixtures. It particularly embraces the racemic forms and the isolated optical isomers having the specified activity.

The term ‘dissolved’, ‘dissolving’, ‘solubilized’ or ‘solubilizing’, means that allantoin or the solubilization enhancer is substantially solubilized in the aqueous vehicle, and that allantoin or the solubilization enhancer will not exist to any appreciable degree in the particulate or crystalline form in the aqueous composition.

The term ‘physically stable’ or ‘physical stability’, when used in accordance with the present invention, means physical or solubilization stability of the composition, rather than chemical stability. The allantoin compositions are considered to be physically stable when substantially no evidence of crystal formation or precipitation is evident after they are stored at room temperature for at least 7 days.

The term ‘room temperature’, as used herein, means a temperature of from about 18.degree. C. to about 25.degree. C.

A ‘solubilization enhancer’ is a pharmaceutically acceptable chemical compound or a suitable combination of such compounds that when present in a solvent, increases the solubility of a second chemical compound, such as an active ingredient, in the solvent.

As used herein, the term ‘about’ will be understood by persons of ordinary skill in the art and will vary to some extent on the context in which is used. If there are uses of the term which are not clear to persons of ordinary skill in the art given the context in which is used, ‘about’ will mean up to plus or minus 10% of the particular term.

The term ‘pharmaceutically acceptable’, as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Allantoin is a polar, heterocyclic organic compound with chemical formula: C₄H₆N₄O₃ and molecular weight of 158.12 [chemical name: (2,5-dioxo-4-imidazolidinyl) urea; or 5-ureidohydantoin]. It is a product of purine metabolism. Racemic forms of allantoin are generally obtained from chemical synthesis. Optically pure forms of allantoin can be obtained by extraction procedures. Allantoin is not chemically stable under basic conditions due to hydrolysis of the amide bonds present in allantoin molecule.

The dissolved allantoin may be present in an amount of at least 0.5% by weight, at least 0.75% by weight, at least 1% by weight, or even as much as 5% by weight. Preferably the dissolved allantoin should be present in an amount of 0.5 to 2% by weight.

The solubilization enhancers in accordance with the present invention are organic compounds whose presence can increase the amount of allantoin solubilized in the aqueous vehicles when compared to the amount of allantoin that would be soluble in the aqueous vehicles in the absence of the solubilization enhancers. Preferably the solubilization enhancers are safe, chemically stable, pharmaceutically acceptable organic compounds, or combinations of such compounds. They should also be chemically compatible with other ingredients present in the aqueous compositions.

We have unexpectedly found that urea and urea derivatives are the suitable solubilization enhancers. Urea, a diamide of carbonic acid, is a polar organic compound. Urea has the general formula of H.sub.2 N—C(O)—NH.sub.2. Urea is widely used as a moisturizing compound and/or keratolytic agent in cosmetic, dermatological, and pharmaceutical applications.

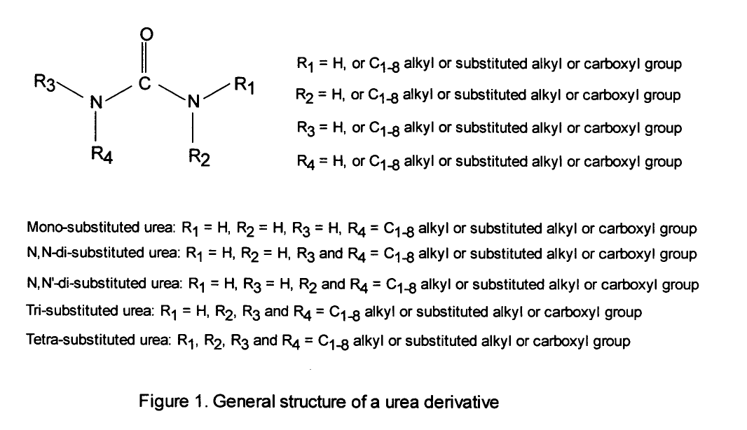

Urea derivatives are derived from urea by substituting one or more of the hydrogen atoms in urea molecule with any suitable chemical groups. The urea derivatives, as used herein, have the general formula of R.sub.3 R.sub.4 N—C(O)—NR.sub.1 R.sub.2, where R.sub.1, R.sub.2, R.sub.3, and R.sub.4 are each independently hydrogen or C.sub.1-8 alkyl or substituted alkyl group or carboxyl group. The alkyl group can be a straight or branched chain alkyl or a cycloalkyl group. Chemical groups such as, for example, hydroxyl, ether or halogen, can be substituted onto the alkyl chain to give the substituted alkyl group. It is possible to have multiple substitutions on a single alkyl group. Examples of the suitable substituted alkyl group include, but not limited to, hydroxyalkyl, ether, or halogen group. Ether group has a general chemical structure of —OR, where R is a suitable alkyl group. The halogen group consists of fluoro-, chloro-, and bromo-group. The carboxyl group can be aliphatic or aromatic carboxyl group. The general structure of a urea derivative is shown in FIG. 1.

All physical forms of the urea derivatives, crystalline, semi-crystalline, and amorphous, are contemplated and within the scope of the present invention, either in admixture or in pure or substantially pure form. Furthermore, all stereoisomers of the urea derivatives are also contemplated and within the scope of the present invention. The definition of the urea derivatives in accordance with the present invention embraces all possible stereoisomers and their mixtures. It particularly embraces the racemic forms and the isolated optical isomers having the specified activity. The racemic forms are mixtures of the optically pure isomers. The individual optical isomers can be obtained from either asymmetric chemical synthesis or chiral separation methods such as, for example, chiral column chromatography, and co-crystallization with optically pure compounds.

When one hydrogen atom in urea molecule is substituted by an alkyl, a substituted alkyl or carboxyl group, a mono-substituted urea is formed. When two hydrogen atoms in urea molecule are substituted by alkyl, substituted alkyl or carboxyl groups, a di-substituted urea is formed. There are two types of di-substituted urea: N,N-di-substituted and N,N′-di-substituted urea. When three hydrogen atoms in urea molecule are substituted by alkyl, substituted alkyl or carboxyl groups, a tri-substituted urea is formed. When all four hydrogen atoms in urea molecule are substituted by alkyl, substituted alkyl or carboxyl groups, a tetra-substituted urea is formed.

Examples of the suitable di-substituted urea are N,N-dimethyl urea, N,N′-dimethyl urea, N,N-diethyl urea, N,N′-diethyl urea, N-ethyl-N′-2-hydroxyethyl urea, N,N′-bis-(1-butyl) urea, N,N′-bis-(2-hydroxyethyl) urea, N,N-bis-(2-hydroxyethyl) urea, N,N′-bis-(3-hydroxypropyl) urea, N,N-bis-(2-hydroxypropyl) urea, N,N′-bis-(2-hydroxypropyl) urea, N,N′-bis-(4-hydroxybutyl) urea, N,N-diacetyl urea, and N,N′-diacetyl urea.

Examples of the suitable tri-substituted urea are N,N-bis-(2-hydroxypropyl)-N′-(2-hydroxyethyl) urea, N,N-bis-(2-hydroxyethyl)-N′-methyl urea, N,N-bis-(2-hydroxyethyl)-N′-ethyl urea, N,N-dimethyl-N′-(2-hydroxyethyl) urea, N,N-diethyl-N′-(2-hydroxyethyl) urea, and N,N-bis-(2-hydroxyethyl)-N′-propyl urea.

Examples of the suitable tetra-substituted urea are N,N,N′,N′-tetramethyl urea, N,N,N′,N′-tetrakis-(1-butyl) urea, N,N,N′,N′-tetrakis-(2-hydroxyethyl) urea, N,N,N′,N′-tetrakis-(2-hydroxypropyl) urea, and N,N-bis-(2-hydroxyethyl)-N′,N′-dimethyl urea.

Among the substituted ureas, mono-substituted ureas are preferred solubilization enhancers. The mono-substituted ureas comprise mono-substituted alkyl ureas, mono-substituted hydroxyalkyl ureas, and mono-substituted carboxyl ureas.

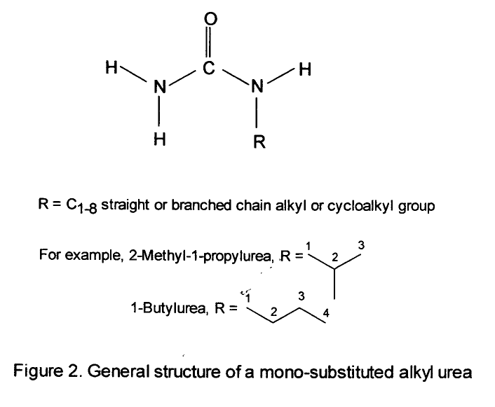

The mono-substituted alkyl ureas have the general structure as shown in FIG. 2. The alkyl group can be a straight or branched chain alkyl, or a cycloalkyl group. Examples of the suitable mono-substituted alkyl ureas are methyl urea, ethyl urea, 1-propyl urea, 2-propyl urea, 1-butyl urea, 2-butyl urea, 1-pentyl urea, 1-hexyl urea, 2-methyl-1-propyl urea, cyclohexyl urea, and combinations thereof.

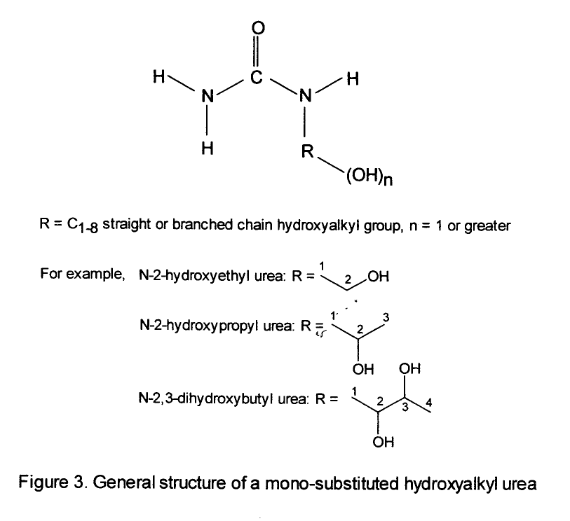

The mono-substituted hydroxyalkyl ureas have the general structure as shown in FIG. 3. The alkyl group can be a straight or branched chain alkyl group with one or more hydroxyl groups attached onto the alkyl chain at any suitable positions. Examples of the suitable mono-substituted hydroxyalkyl ureas are N-2-hydroxyethyl urea, N-3-hydroxypropyl urea, N-2-hydroxypropyl urea, N-2,3-dihydroxypropyl urea, N-4-hydroxybutyl urea, N-3-hydroxybutyl urea, N-2-hydroxybutyl urea, N-2,3-dihydroxybutyl urea, N-2,4-dihydroxybutyl urea, N-3,4-dihydroxybutyl urea, and combinations thereof.

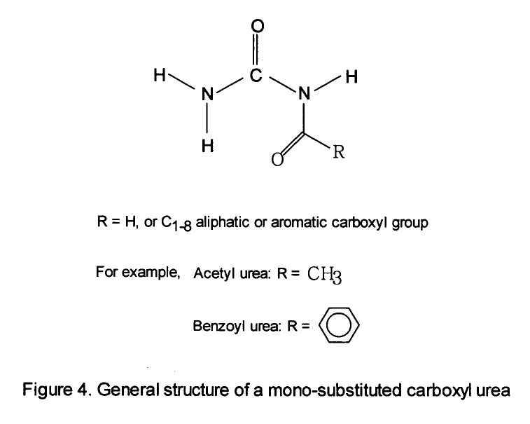

The mono-substituted carboxyl ureas have the general structure as shown in FIG. 4. The carboxyl group can be aliphatic or aromatic. Examples of the suitable mono-substituted carboxyl ureas are formyl urea, acetyl urea, propionyl urea, butyryl urea, benzoyl urea, and combinations thereof.

In addition to their function as the solubilization enhancers, the solubilized urea derivatives can also bring cosmetic and therapeutic functions to the aqueous compositions. For example, N-2-hydroxyethyl urea is also an excellent moisturizer. 1-Butyl urea has antimicrobial activity.

Combinations of urea, alkyl ureas, hydroxyalkyl ureas, or carboxyl ureas in any given ratio are also suitable as the solubilization enhancers.

Urea is most preferred solubilization enhancer. Urea is a well known moisturizer. High concentrations of urea, such as greater than 40%, are known to have keratolytic activity as well as mild, antimicrobial effect. Thus, the dissolved urea in the compositions serves not only as the solubilization enhancer but also as a moisturizer and keratolytic agent.

Allantoin is also known to have keratolytic activity. This combination of urea and allantoin may produce a synergistic keratolytic efficacy.

The dissolved urea or urea derivatives may be present in an amount of at least 10% by weight, at least 20% by weight, at least 30% weight, or even as much as 55% by weight. Preferably the dissolved solubilization enhancers should be present in an amount of about 20% to about 50% by weight.

One method for dissolving allantoin in the aqueous vehicle using the solubilization enhancer is to dissolve the solubilization enhancer first in the aqueous vehicle before allantoin is added. The mixture is maintained at a temperature from room temperature to about 80.degree. C. while stirring until allantoin is dissolved. Then, the solution is cooled to room temperature.

Another method for dissolving allantoin is to add the solubilization enhancer and allantoin together to the aqueous vehicle. The mixture is maintained at a temperature from room temperature to about 80.degree. C. while stirring until allantoin and the solubilization enhancer are dissolved. Then, the solution is cooled to room temperature.

Preferred solubilization temperature is a temperature from room temperature to about 50.degree. C.

The aqueous composition, in accordance with the present invention, may be in the form of a solution, spray or gel. Preferably the composition is a gel. Therefore, the aqueous allantoin composition preferably contains a gelling agent. Any gelling agent that is dispersible in the aqueous vehicle and forms an aqueous gel of substantially uniform consistency is suitable for use in the present invention. The gelling agent should not substantially decrease the solubility of allantoin in the aqueous vehicle or reduce the therapeutic efficacy of the composition. “Substantially decrease” means that the inclusion of the gelling agent decreases the solubility of allantoin to 0.5% by weight or less in the composition.

Examples of the suitable gelling agents are polycarbohydrate based gelling agents and polyacrylic acid based gelling agents. Examples of the suitable polycarbohydrate gelling agents are hydroxyethylcellulose, hydroxypropylcellulose, and xanthan gum. Examples of the suitable polyacrylic acid gelling agents are CARBOPOL Brand 934, 940, 941, Ultrez 10, and Ultrez 20 (available from Noveon Corp., Cleveland, Ohio). Combinations of the polycarbohydrate gelling agents and polyacrylic acid gelling agent are also suitable as the gelling agents.

Polyacrylic acid gelling agents are high molecular weight homo- and copolymers of acrylic acid crosslinked with suitable crosslinkers, such as, for example, polyalkenyl polyether. They need to be neutralized by basic neutralizing agents to achieve their rheological features and performance properties. Since allantoin and urea degrade under basic conditions (i.e., chemically unstable), the pH of the aqueous composition after neutralization should be in the range of from about 3.0 to about 6.5, more preferably from about 3.0 to about 6.0, even more preferably from about 3.0 to about 5.5. Inorganic basic compounds, such as sodium hydroxide, potassium hydroxide, or ammonium hydroxide, are suitable neutralizing agents. Organic basic compounds are preferred neutralizing agents. Examples of the suitable organic basic compounds are aminoethyl propanol, triethanol amine, diisopropanol amine, triisopropanol amine, and tetrahydroxypropyl ethylenediamine.

The aqueous allantoin composition of the present invention can contain conventional amounts of moisturizing compounds: polyhydric alcohols (also known as polyols), polyol ethers and esters, low molecular weight polyethylene glycols, lactates, sugars, methyl glucose esters, sodium pyrrolidone carboxylic acid, sodium hyaluronate, hyaluronic acid, alpha.- and beta.-hydroxy acids. Examples of the suitable polyols are glycerin (also known as glycerol), propylene glycol (also known as 1,2-propanediol), 1,3-propanediol, 1,2-butanediol, 1,3-butanediol, 1,4-butanediol, 2,3-butanediol, 1,2-pentanediol, 1,5-pentanediol, 1,2-hexanediol, 2-methyl-2,4,-pentanediol (also known as hexylene glycol), 1,2-hexanediol, 1,6-hexanediol, diethylene glycol, diglycerin, dipropylene glycol, triethylene glycol, 1,2,3-hexanetriol, 1,2,6-hexanetriol, or combinations of the suitable polyols in any given ratio. Preferred polyols are glycerin, propylene glycol, 1,4-butanediol, and hexylene glycol. Examples of the suitable low molecular weight polyethylene glycols (PEG) are PEG 200, PEG 300, PEG 400, and PEG 600 (The number after PEG indicates average molecular weight of a PEG). Examples of the suitable lactates are ammonium lactate, sodium lactate, and potassium lactate. Examples of the suitable methyl glucose esters are methyl gluceth-10 and methyl gluceth-20.

The aqueous allantoin composition of the present invention can also contain conventional amounts of one or more other desirable ingredients: vitamins, coenzymes, skin penetration enhancers, emulsifiers, emollients, herbal extracts, chelating agents, antibiotics, colorants, antioxidants, and even sunscreens. Examples of the suitable desirable ingredients are: ascorbic acid (vitamin C) and derivatives, grape seed extract, water soluble vitamin E derivatives (e.g., vitamin E phosphate), retinol, retinoic acids, vitamin A palmitate, vitamin K and derivatives, silicone-polyol copolymer emulsifiers, or propyl gallate. Examples of the suitable chelating agents are EDTA (ethylenediaminetetraacetic acid) and EGTA [ethylenebis(oxyethylenenitrilo)tetraacetic acid] and their pharmaceutically acceptable salts.

The following examples are included for purposes of illustrating the technology covered by this disclosure. They are not intended to limit the scope of the claimed invention in any manner. One skilled in the art will understand that there are alternatives to these specific embodiments that are not completely described by these examples.

### EXAMPLE 1

This example is to demonstrate the solubilization enhancing effect of urea in water at 1% allantoin concentration. An aqueous composition in accordance with the invention was prepared as follows:

| Component | Amount (weight percentage) |
|---|---|
| Allantoin | 1% |
| Urea | 10% |
| Water | 89% |

Urea and allantoin were added together to water at room temperature. The mixture was kept at room temperature while stirring until urea and allantoin were completely dissolved. The solution was physically stable for at least 7 days.

### EXAMPLE 2

This example is to demonstrate the solubilization enhancing effect of urea in water at 2% allantoin concentration. An aqueous composition in accordance with the invention was prepared as follows:

| Component | Amount (weight percentage) |
|---|---|
| Allantoin | 2% |
| Urea | 43% |
| Water | 55% |

Urea and allantoin were added together to water at room temperature. The mixture was kept at room temperature while stirring until urea and allantoin were completely dissolved. The solution was physically stable for at least 7 days.

### EXAMPLE 3

This example is to demonstrate the solubilization enhancing effect of methyl urea, a mono-substituted alkyl urea, in water. An aqueous composition in accordance with the invention was prepared as follows:

| Component | Amount (weight percentage) |
|---|---|
| Allantoin | 1% |
| Methyl urea | 30% |
| Water | 69% |

Methyl urea and allantoin were added together to water at room temperature. The mixture was kept at room temperature while stirring until methyl urea and allantoin were completely dissolved. The solution was physically stable for at least 7 days.

### EXAMPLE 4

This example is to demonstrate the solubilization enhancing effect of N-2-hydroxyethyl urea, a mono-substituted hydroxyalkyl urea, in water. An aqueous composition in accordance with the invention was prepared as follows:

| Component | Amount (weight percentage) |
|---|---|
| Allantoin | 1% |
| N-2-hydroxyethyl urea | 30% |
| Water | 69% |

N-2-hydroxyethyl urea and allantoin were added together to water at room temperature. The mixture was kept at room temperature while stirring until N-2-hydroxyethyl urea and allantoin were completely dissolved. The solution was physically stable for at least 7 days.

### EXAMPLE 5

This example is to demonstrate the solubilization enhancing effect of a mixture of urea and N-2-hydroxyethyl urea in water. An aqueous composition in accordance with the invention was prepared as follows:

| Component | Amount (weight percentage) |
|---|---|
| Allantoin | 1.5% |
| N-2-hydroxyethyl urea | 10% |
| Urea | 30% |
| Water | 58.5% |

N-2-hydroxyethyl urea and urea were dissolved in water at 45.degree. C. Allantoin was added to the solution at 45.degree. C. The mixture was maintained at 45.degree. C., while stirring until allantoin was completely dissolved. The solution was allowed to cool to room temperature. The solution was physically stable for at least 7 days.

### EXAMPLE 6

This example is to demonstrate the solubilization enhancing effect of urea in water and the composition is in the form of a gel using a polyacrylic acid gelling agent. An aqueous composition in accordance with the invention was prepared as follows:

| Component | Amount (weight percentage) |
|---|---|
| Allantoin | 1% |
| Urea | 40% |
| PEG 400 | 10% |
| Water | 48.5% |
| Ultrez 10 | 0.5% |

Urea was dissolved in water and PEG 400 mixture at 45.degree. C. Allantoin was added to the solution at 45.degree. C. The mixture was maintained at 45.degree. C., while stirring until allantoin was completely dissolved. The solution was allowed to cool to room temperature. Then, a polyacrylic acid gelling agent, Ultrez 10 (available from Noveon Corp., Cleveland, Ohio), was dispersed into the solution. After Ultrez 10 was completely wetted, the mixture was neutralized by triethanol amine to pH of about 5.5, while mixing until the mixture turned into a clear gel of uniform consistency. The final product was physically stable for at least 7 days.

### EXAMPLE 7

This example is to demonstrate the solubilization enhancing effect of urea in water and the composition is in the form of a gel using a polycarbohydrate gelling agent. An aqueous composition in accordance with the invention was prepared as follows:

| Component | Amount (weight percentage) |
|---|---|
| Allantoin | 1.5% |
| Urea | 40% |
| Propylene glycol | 5% |
| Water | 51.5% |
| Hydroxypropylcellulose | 2% |

Urea was dissolved in water and propylene glycol mixture at 45.degree. C. Allantoin was added to the solution at 45.degree. C. The mixture was maintained at 45.degree. C., while stirring until allantoin was completely dissolved. The solution was then cooled to room temperature. Hydroxypropylcellulose (available from Hercules Inc., Wilmington, Del.) was added to the solution. With stirring, hydroxypropylcellulose was completely solubilized. The final product was a clear gel of uniform consistency and physically stable for at least 7 days.

It will thus be seen that the objects set forth above, among those made apparent from the preceding description, are efficiently attained and, since certain changes may be made in carrying out the above process and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible mixtures of such ingredients wherever the sense permits.

What is claimed is:

1. An aqueous composition comprising, by weight of the total composition:
allantoin, in an amount of from about 0.5% to about 5%,
a solubilization enhancer selected from the group consisting of urea, urea derivatives, and combinations thereof, in an amount of from about 10% to about 55%,
wherein the solubilization enhancer and allantoin are substantially solubilized in the aqueous composition and the composition is physically stable.

2. The composition of claim 1 wherein the solubilization enhancer comprises mono-substituted urea.

3. The composition of claim 2 wherein the mono-substituted urea is mono-substituted alkyl urea.

4. The composition of claim 2 wherein the mono-substituted urea is mono-substituted hydroxyalkyl urea.

5. The composition of claim 2 wherein the mono-substituted urea is mono-substituted carboxyl urea.

6. The composition of claim 1 wherein the solubilization enhancer is urea.

7. The composition of claim 1 which is in the form of a solution, spray or gel.

8. The composition of claim 6 which is a gel.

9. A method for enhancing solubility of allantoin in an aqueous vehicle using a solubilization enhancer selected from the group consisting of urea, urea derivatives, and combinations thereof, comprising:
(a) combining allantoin and the solubilization enhancer in an aqueous fluid,
(b) subjecting the combination from (a) to a temperature of from room temperature to about 80.degree. C. for sufficient time to permit allantoin and the solubilization enhancer to dissolve,
wherein the concentration of the solubilization enhancer is from about 10% by weight to about 55% by weight and the concentration of allantoin is about 0.5% by weight or higher.

10. The method of claim 9 wherein the solubility of allantoin is increased to about 0.5% by weight or more.

11. The method of claim 9 wherein the solubility of allantoin is increased to about 1.0% by weight or more.

12. The method of claim 9 wherein the solubilization enhancer is urea.

13. The method of claim 9 wherein the solubilization enhancer comprises mono-substituted urea.

14. The method of claim 13 wherein the mono-substituted urea is selected from the group consisted of mono-substituted alkyl urea, mono-substituted hydroxyalkyl urea, and mono-substituted carboxyl urea.

15. A method for obtaining an aqueous composition containing solubilized allantoin at a level of 0.5% by weight or higher, comprising:
(a) combining allantoin and a solubilization enhancer selected from the group consisted of urea, urea derivatives, and combinations thereof, in an amount of from about 10% by weight to about 55% by weight, in an aqueous liquid,
(b) subjecting the combination in (a) to a temperature of from room temperature to about 80.degree. C. for sufficient time to permit allantoin and the solubilization enhancer to dissolve to form the aqueous allantoin composition,
wherein allantoin and the solubilization enhancer are substantially solubilized and the aqueous composition is physically stable.

16. The method of claim 15 wherein the solubilization enhancer is urea.

17. The method of claim 15 wherein the aqueous composition is in the form of a solution, spray or gel.

18. The method of claim 16 wherein the aqueous composition is a gel.

19. The method of claim 15 wherein the solubilization enhancer comprises mono-substituted urea.

20. The method of claim 19 wherein the mono-substituted urea is selected from the group consisted of mono-substituted alkyl urea, mono-substituted hydroxyalkyl urea, and mono-substituted carboxyl urea.
